(19) United States
(12) Patent Application Publication
(10) Pub. No.: US 2026/0207846 A1
(43) Pub. Date: Jul. 23, 2026

(54) Splittable Needle for Catheter Placement System

(71) Applicant: Bard Peripheral Vascular, Inc., Franklin Lakes, NJ (US)

(72) Inventors: Bryon Ray Densley, Oklahoma City, OK (US); Brenda L. F. Fedor, Holladay, UT (US); Diana Fiumefreddo, Salt Lake City, UT (US); Danielle Mariah Pompa, Bountiful, UT (US); Ian N. Thomas, West Bountiful, UT (US)

(21) Appl. No.: 19/145,086

(22) PCT Filed: Jan. 4, 2023
(86) PCT No.: PCT/US2023/010119

§ 371 Date: Jul. 1, 2025

(51) Int. Cl. A61M 5/19; A61M 5/178; A61M 5/31

(52) U.S. Cl. CPC A61M 5/19; A61M 5/3146; A61M 2005/1787

(57) ABSTRACT

Intravenous catheter patients are required to visit a clinic at regular intervals to have their implanted vascular access device flushed for maintenance. This can be costly in both time and resources for both the patient and the clinic. Flushing the device using standard equipment requires specialized training to prevent over-pressurization of the device. Embodiments include a flushing system including a first syringe chamber for aspirating a blood flow, confirming vascular access, and confirming the device is occlusion free. Once confirmed, the device automatically unlocks a flushing syringe chamber to allow the patient to flush the device. The user can flush the device with little to no training and prevents over-pressurization of the implanted vascular access device.

### BACKGROUND

Current standards of care for intravenous catheter patients require the patient to visit a clinic at regular intervals to have their implanted device “flushed” for maintenance. For example for implantable ports, typically the device needs to be flushed every 30 days or so, however, greater or lesser intervals can also occur depending on the device and other circumstances. As will be appreciated, these maintenance appointments can be inconvenient, time consuming, and costly not only for the patient but also to the clinicians who must dedicate resources to performing these maintenance appointments.

Using standard syringe and access needle equipment requires a skilled user to access the device, confirm vascular access, and confirm the device is not occluded, before administering the saline flush fluids. Administering the flushing fluids without correctly confirming vascular access and the device is occlusion free can lead to over pressurization of the implanted device, causing damage to the device, and may require replacement of the device.

### SUMMARY

Embodiments include a flushing device that can automatically confirm vascular access and confirm an occlusion-free fluid pathway before unlocking the saline flush actuator. This can allow a user with little to no training, or the patient themselves, to administer saline flush in a timely manner and prevent the user from accidentally over-pressurizing the device. As such, the patient can receive the maintenance flush in the comfort of their own home saving time and resources for both the patient and the clinic.

Disclosed herein is a flushing system for a vascular access device including, a body defining a first chamber and a second chamber, the second chamber pre-filled with a flushing fluid, a nozzle disposed at a distal end of the body and in fluid communication with the vascular access device, the nozzle defining a first conduit in fluid communication with the first chamber, and a second conduit in fluid communication with the second chamber, a first plunger slidably engaged with the first chamber and configured to be withdrawn proximally to draw a fluid from the first conduit into the first chamber, a second plunger slidably engaged with the second chamber and configured to urge the flushing fluid through the second conduit, and a locking mechanism including a pawl engaged with the second plunger in a locked position and configured to inhibit movement thereof, the locking mechanism configured to transition to an unlocked position when the fluid fills at least a portion of the first chamber.

In some embodiments, the locking mechanism further includes an aperture disposed proximally of the portion of the first chamber and communicating with a well, defined by the body, the aperture configured to provide a fluid flow from the first chamber into the well that causes the pawl to disengage from the second plunger.

In some embodiments, the fluid flow within the well causes the pawl to disengage the second plunger under a gravity flow.

In some embodiments, the locking mechanism further includes a float actuator configured to transition to an unlocked position when the fluid from the first chamber enters the well.

In some embodiments, the float actuator includes a one or more of a balloon, air space, or second material that is less dense than the fluid within the first chamber.

In some embodiments, the float actuator is pivotably coupled to an inner surface of the well and configured to pivot between the locked position and the unlocked position.

In some embodiments, the locking mechanism further includes a pushrod slidably engaged with the well and transitionable between a locked position and an unlocked position, in the locked position, the pushrod engages the pawl and prevents the pawl from disengaging the second plunger, in the unlocked position the pushrod disengages the pawl to allow the pawl to disengage the second plunger.

In some embodiments, the float actuator in the locked position engages the pushrod to prevent the pushrod from transitioning to the unlocked position, and the float actuator in the unlocked position allows the pushrod to slide to the unlocked position.

Also disclosed is a method of flushing an implanted catheter including, withdrawing a first plunger, slidably engaged with a first chamber of a body, to draw a predetermined volume of fluid through a nozzle and into the first chamber, transitioning a locking mechanism to an unlocked position, using the fluid within the first chamber, to disengage the locking mechanism from a second plunger, and sliding the second plunger distally to urge a flushing solution that is prefilled within a second chamber of the body through the nozzle and into the implanted catheter.

In some embodiments, subsequent to the predetermined volume of fluid entering the first chamber, flowing a portion of the fluid from the first chamber into a well, defined by the body, to actuate the locking mechanism and disengage a pawl from the second plunger.

In some embodiments, the portion of the fluid from the first chamber actuates the pawl under a gravity flow.

In some embodiments, the portion of the fluid within the well actuates a float actuator from a locked position to an unlocked position.

In some embodiments, the float actuator includes a portion that is less dense than the portion of the fluid within the well.

In some embodiments, the method further includes transitioning the float actuator and a pushrod to an unlocked position that transitions the pawl to the unlocked position.

In some embodiments, the fluid in the first chamber is blood and the flushing solution in the second chamber includes one of water, saline solution, or Ringers solution.

In some embodiments, the nozzle defines a first conduit in fluid communication with the first chamber and a second conduit in fluid communication with the second chamber.

In some embodiments, the nozzle is configured to engage an access needle that is configured to provide fluid communication with the implanted catheter.

Also disclosed is a catheter and flushing system including, a catheter extending between a proximal end and a distal tip, and a flushing system including, a body defining a first chamber and a second chamber, a first plunger slidably engaged with the first chamber, a second plunger slidably engaged with the second chamber, the second chamber prefilled with a flushing solution, and a locking mechanism configured, in the locked position, to prevent the second plunger from moving, and configured to transition to an unlocked position to allow the second plunger to move when a predetermined volume of fluid is disposed within the first chamber.

In some embodiments, the predetermined volume of fluid within the first chamber is greater than a volume disposed within the catheter between the proximal end and the distal tip.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a schematic view of a gravity actuated flushing system in a locked configuration, in accordance with embodiments disclosed herein.

FIG. 2 shows a schematic view of the gravity actuated flushing system of FIG. 1 in an unlocked configuration, in accordance with embodiments disclosed herein.

FIG. 3 shows a schematic view of a float actuated flushing system in a locked configuration, in accordance with embodiments disclosed herein.

FIG. 4 shows a schematic view of the float actuated flushing system of FIG. 3 in an unlocked configuration, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a needle disclosed herein includes a portion of the needle intended to be near a clinician when the needle is used on a patient. Likewise, a “proximal length” of, for example, the needle includes a length of the needle intended to be near the clinician when the needle is used on the patient. A “proximal end” of, for example, the needle includes an end of the needle intended to be near the clinician when the needle is used on the patient. The proximal portion, the proximal end portion, or the proximal length of the needle can include the proximal end of the needle; however, the proximal portion, the proximal end portion, or the proximal length of the needle need not include the proximal end of the needle. That is, unless context suggests otherwise, the proximal portion, the proximal end portion, or the proximal length of the needle is not a terminal portion or terminal length of the needle.

With respect to “distal,” a “distal portion” or a “distal end portion” of, for example, a needle disclosed herein includes a portion of the needle intended to be near or in a patient when the needle is used on the patient. Likewise, a “distal length” of, for example, the needle includes a length of the needle intended to be near or in the patient when the needle is used on the patient. A “distal end” of, for example, the needle includes an end of the needle intended to be near or in the patient when the needle is used on the patient. The distal portion, the distal end portion, or the distal length of the needle can include the distal end of the needle; however, the distal portion, the distal end portion, or the distal length of the needle need not include the distal end of the needle. That is, unless context suggests otherwise, the distal portion, the distal end portion, or the distal length of the needle is not a terminal portion or terminal length of the needle.

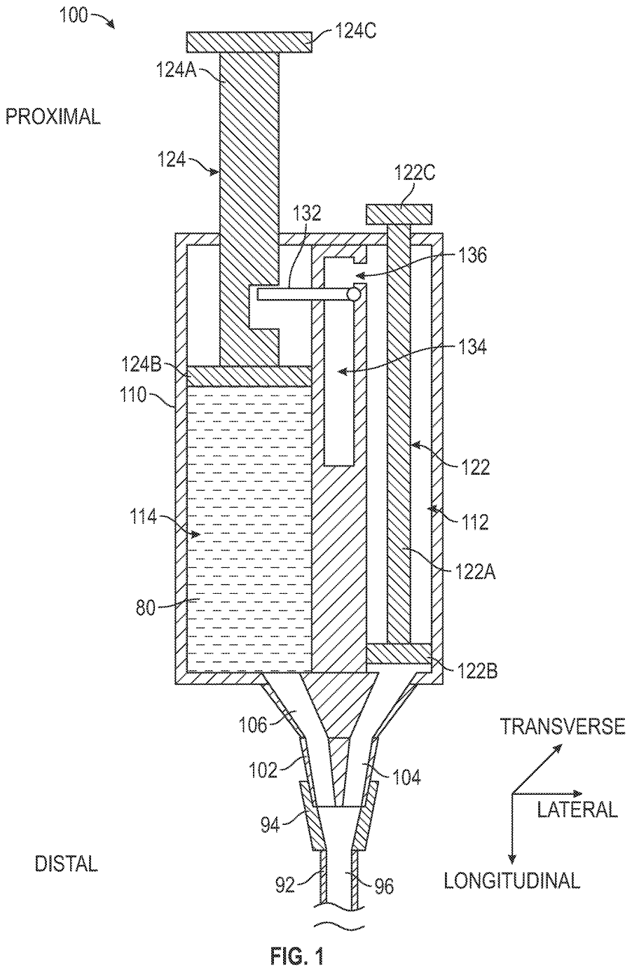

To assist in the description of embodiments described herein, as shown in FIG. 1, a longitudinal axis extends substantially parallel to an axial length of the needle. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

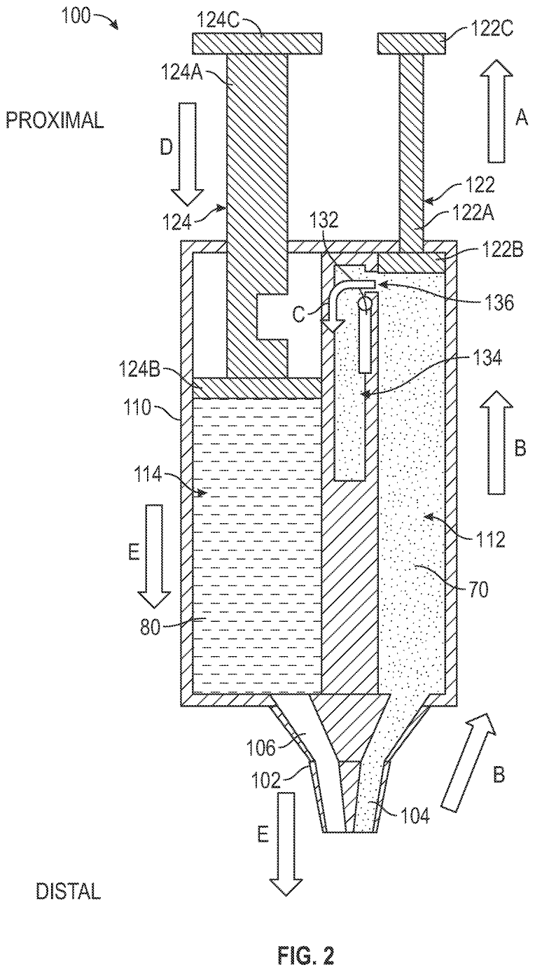

FIGS. 1-2 show a schematic view of a gravity actuated flushing system (“system”) 100. The system 100 generally includes a nozzle 102 and a body 110. The nozzle is configured to engage an access needle 92 and provide fluid communication therewith. For example, the nozzle 102 engages a needle hub 94 using a luer lock, spin nut, bayonet fitting, or similar engagement structure. One or more conduits of the nozzle 102 (e.g. a first conduit 104 and a second conduit 106) can be in fluid communication with the needle lumen 96.

The needle 92 in turn accesses a vascular access device such as a port, subcutaneous port, extension leg, fluid connector, valve, combinations thereof or the like, to access a vasculature. In an embodiment, the needle 92 accesses a vasculature directly. In an embodiment, the needle 92 includes two or more lumens. A first lumen of the needle 92 can be in fluid communication with a first conduit 104 of the nozzle 102. Similarly, a second lumen of the needle 92 can be in fluid communication with a second conduit 106 of the nozzle 102. These and other combinations of one or more needle lumen and one or more nozzle conduits are contemplated to fall within the scope of the present invention.

In an embodiment, the body 110 defines a first chamber 112 and a second chamber 114. The first chamber 112 is in fluid communication with the first conduit 104, and the second chamber 114 is in fluid communication with the second conduit 106. The system 110 further includes a first plunger 122 slidably engaged with the first chamber 112 between a distal position and a proximal position. The system 110 also includes a second plunger 124 slidably engaged with the second chamber 114 between a distal position and a proximal position. Each of the first plunger 122 and the second plunger 124 includes a shaft 122A, 124A extending longitudinally through the chamber and includes a plunger head 122B, 124B disposed at a distal end and a plunger handle 122C, 124C disposed at a proximal end. The plunger head 122B, 124B slidably engages a wall of the chamber 112, 114 to form a fluid tight seal therebetween while still allowing the plunger 122, 124 to slide back and forth through the chamber.

In an embodiment, as shown in FIG. 1 and prior to being used, the system 100 is provided including the first plunger 122 in the distal position and the first chamber 112 being substantially empty. The second plunger 124 is in the proximal position and is pre-filled with a flushing solution 80. Exemplary flushing solutions 80 include water, saline, Ringers solution, combinations thereof, or the like. Optionally, the flushing solution 80 includes one or more active ingredients such as heparin, citrate, combinations thereof, or the like.

In an embodiment, the system 100 includes a locking mechanism 130 transition between a locked position and an unlocked position. As shown, in the locked position, the locking mechanism 130 is configured to engage the second plunger 124 and prevent any distal movement of the second plunger 124. In the unlocked position, the locking mechanism 130 allows the second plunger 124 to slide through the second chamber 114 between the proximal position and the distal position, as described in more detail herein.

In an embodiment, as shown in FIGS. 1-2, the locking mechanism 130 is a gravity actuated locking mechanism 130. The locking mechanism 130 includes a pawl 132 pivotably coupled with the body 110 and rotatable between a locked position and an unlocked position. As noted, in the locked position (FIG. 1), the pawl 132 engages the second plunger 124 and prevents the second plunger from moving through the second chamber 114. In the unlocked position (FIG. 2), the pawl 132 disengages the second plunger 124 and allows the second plunger 124 to travel through the second chamber 114. The locking mechanism 130 further includes a well 134 that is in fluid communication with the first chamber 112 by way of an aperture 136 extending between the well 134 and the first chamber 114. In an embodiment, the aperture 136 is disposed proximally of the pawl 132.

In an exemplary method of use, as shown in FIG. 2, a flushing system 100 including a gravity actuated locking mechanism 130 is provided, as described herein. A user couples a needle hub 94 to the nozzle 102 and accesses a vascular access device (VAD), e.g. a subcutaneous port, or the like, to place the needle lumen 96 in fluid communication with the VAD. The VAD, in turn, is in fluid communication with the vasculature of the patient.

As shown in step (A), the user withdraws the first plunger 122 from the distal position to the proximal position. The plunger head 122B of the first plunger 122 maintains a fluid tight seal between the first plunger head 122B and the inner surface of the first chamber 112. As such, a vacuum or low pressure environment is created within a portion of the first chamber 112, distal to the first plunger head 122B and draws a fluid (e.g. blood 70) flow proximally from the vasculature, through the VAD, through the needle lumen 96, through the first conduit 104, and into the first chamber 112. As the blood 70 fills the first chamber 112, the user can observe the blood 70 to confirm vascular access is achieved. To note, at least a portion of the body 110 can be formed of a transparent or translucent material to allow a user to observe the contents of one or both of the first chamber 112 and the second chamber 114.

As shown in step (B), a blood flow 70 continues to fill the first chamber 112 until the first plunger head 122B is disposed proximally of the aperture 136, placing the well 134 in fluid communication with the first chamber 112. As shown in step (C), the blood 70 flows from the first chamber 112 into the well 134 and engages the pawl 132. The weight of the blood fluid 70 engaging the pawl 132 is sufficient to transition the pawl 132 from the locked position to the unlocked position, disengaging pawl 132 from the second plunger 124. In an embodiment, prior to the system 100 being actuated, the well 130 maintains a vacuum therein. As such, as the first plunger head passes to the proximal side of the aperture 136, the vacuum is placed in fluid communication with the first chamber 112 and draws the blood 70 fluid into the well 134 to engage the pawl 132.

Advantageously, a volume of the first chamber 112 between the first conduit 104 and the aperture 136 is designed to be large enough to ensure that a sufficient amount of fluid 70 is drawn into the first chamber 112 before the gravity locking mechanism 130 is actuated. For example, a sufficient amount of fluid 70 is equal to or greater than a volume of fluid contained within the VAD (e.g. between a proximal end and a distal tip thereof) and the needle lumen 96. This ensures the system 100 is in fluid communication with the vasculature, and that there are no occlusions within the VAD preventing fluid communication with the vasculature, before the second plunger 124 is unlocked. By contrast, where only a relatively small amount of blood 70 is drawn into the first chamber 112, this blood may be from only a portion of the VAD, or the needle lumen 96, and is not indicative that the VAD and the system 100 is in fluid communication with the vasculature, nor that the VAD is free of any occlusions.

As shown in step (D), with locking mechanism 130 unlocked, the system 100 confirms to the user that the nozzle 102 is in fluid communication with the vasculature, and that there are no occlusions within the VAD, obstructing fluid flow. In an embodiment, the locking mechanism 130 can further include one or more indicators functionally coupled to the pawl 132 to indicate to the user as to whether the pawl 132 is in the locked or unlocked position. The user then actuates the second plunger 124 (step D) to urge the flushing solution 80 that is pre-filled into the second chamber 114 through the second conduit 106, into the needle lumen 96, and through the VAD, flushing the VAD (step E).

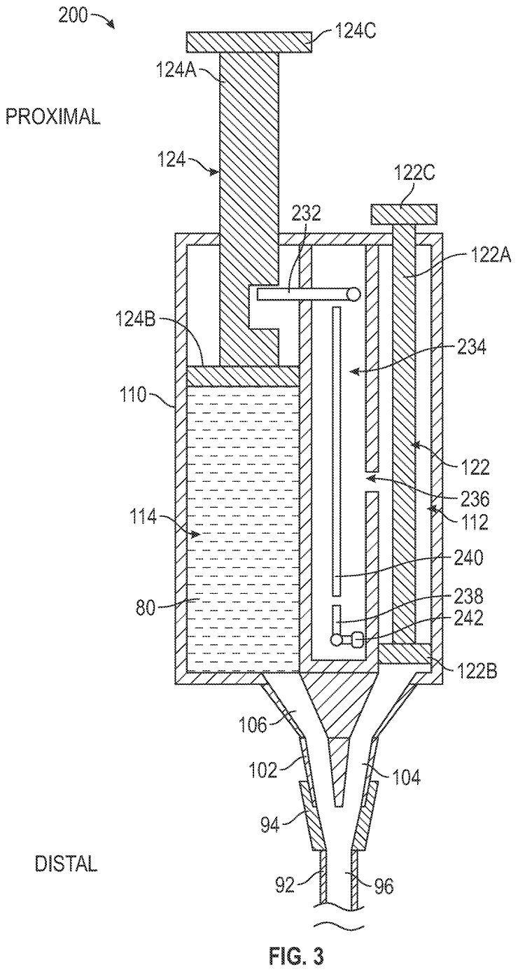

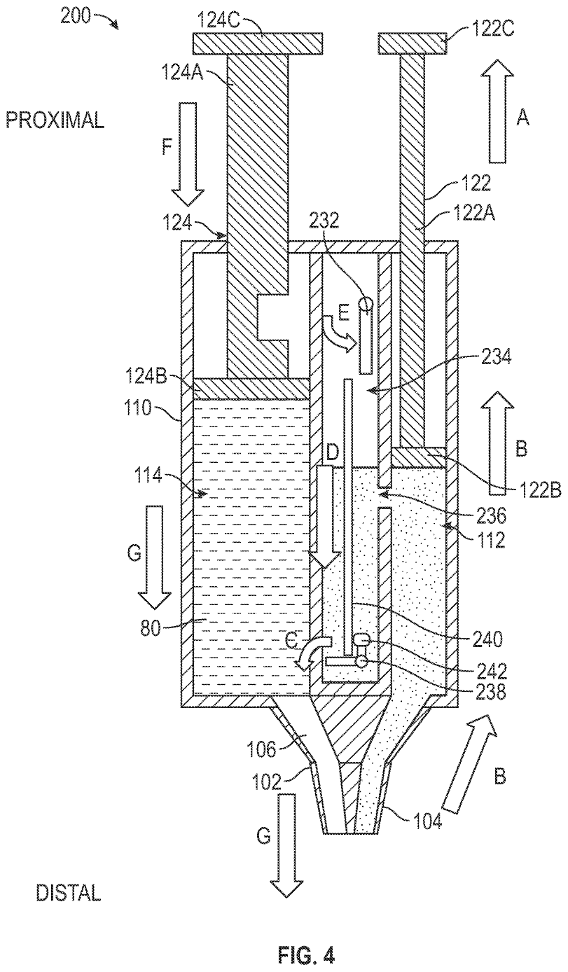

FIGS. 3-4 show an embodiment of a float actuated flushing system (“system”) 200. As described herein, the float actuated system 200 includes a nozzle 102 and a body 110. The body 110 defines a first chamber 112 and a second chamber 114. The nozzle 102 defines a first conduit 104 in fluid communication with the first chamber 112, and a second conduit 106 in fluid communication with the second chamber 114. The first chamber 112 includes a first plunger 122 slidably engaged therewith. The second chamber 114 includes a second plunger 124 slidably engaged therewith. Prior to actuation, the first plunger 112 is disposed in the distal position and the second plunger 124 is disposed in a proximal position. The second chamber 114 is pre-filled with a flushing solution 80, as described herein.

The float actuated flushing system 200 includes a float actuated locking mechanism (“locking mechanism”) 230. The locking mechanism 230 includes a pawl 232, a well 234, an aperture 236, a float actuator (“float”) 238, and a pushrod 240. The pawl 232 is pivotably coupled to the body 110 and transitionable between a locked position and an unlocked position, as described herein. In the locked position, the pawl 232 engages the second plunger 124 and prevents the second plunger 124 from moving through the second chamber 114.

The pushrod 240 is slidably engaged with the well 234 between a locked position and an unlocked position. In the locked position the pushrod 240 prevents the pawl 232 from pivoting between the locked position and the unlocked position. The float 238 is pivotably coupled with the well 232 between a locked position and an unlocked position. In the locked position the float prevents the pushrod 240 from sliding between the locked position and the unlocked position. In an embodiment, the float 238 includes a portion 242, such as a balloon, air bubble, or portion of material that is less dense than the fluid 70.

In an exemplary method of use, as shown in FIG. 4, a flushing system 200 including a float actuated locking mechanism 230 is provided, as described herein. A user couples a needle hub 94 to the nozzle 102 and accesses a vascular access device (VAD), e.g. a subcutaneous port, or the like, to place the needle lumen in fluid communication with the VAD. The VAD is, in turn, in fluid communication with the vasculature of the patient.

As shown in step (A), the user withdraws the first plunger 122 from the distal position to the proximal position. The plunger head 122B of the first plunger 122 maintains a fluid tight seal between the first plunger head 122B and the inner surface of the first chamber 112. As such, a vacuum or low pressure environment is created within a portion of the first chamber 112, distal to the first plunger head 122B that draws a fluid (e.g. blood 70) flow proximally from the vasculature, through the VAD, through the needle lumen 96, through the first conduit 104, and into the first chamber 112. As the blood 70 fills the first chamber 112, the user can observe the blood 70 to confirm vascular access is achieved. To note, at least a portion of the body 110 is formed of a transparent or translucent material to allow a user to observe the contents of one or both of the first chamber 112 and the second chamber 114.

As shown in step (B), a blood flow 70 continues to fill the first chamber 112 until the plunger head is disposed proximally of the aperture 236, placing the well 234 in fluid communication with the first chamber 112. A volume of the first chamber 112 between the first conduit 104 and the aperture 236 can be of a sufficient volume to confirm that the system 200 is in fluid communication with the vasculature and that the VAD is occlusion-free, as described herein. As shown in step (C), the blood 70 flows from the first chamber 112 into the well 134 and fills the well 234, as well as the first chamber 112. The fluid within the well 234 causes the float 238, which includes the float portion 242 at one end, to pivot from the locked position to the unlocked position.

In an embodiment, as shown in step (D), with the float 238 in the unlocked position, this in turn allows the pushrod 240 to slide to the unlocked position, which in turn, as shown in step (E) allows the pawl 232 to transition to the unlocked position and disengage the second plunger 124. In an embodiment, the pushrod 240 in the unlocked position disengages the pawl 232 to allow the pawl 232 to disengage the second plunger 124. Worded differently, one or more of the float 238, pushrod 240 and pawl 232 in the unlocked position can move independently, whereas in the locked position at least the pushrod 240 and the pawl 232 are prevented from moving.

Alternatively, one or more of the float 238, pushrod 240 and pawl 232 remain mechanically engaged and transition between the locked and unlocked positions, i.e. the float 238 transitioning to the unlocked position causes the pushrod 240 and pawl 232 to transition to the unlocked position. For example, as the pushrod 240 transitions to the unlocked position, the pushrod 240 can cause the pawl 232 to disengage the second plunger 124. Similarly, as the float transitions from the locked position to the unlocked position, the float 238 can cause the pushrod 240 to transition to the unlocked position.

As shown in step (F), with the locking mechanism 230 in the unlocked position, the user actuates the second plunger 124 to urge the flushing fluid 80 through the second conduit 106, (step G) through the needle lumen 96 and through the VAD, flushing the VAD.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A flushing system for a vascular access device, comprising:
a body defining a first chamber and a second chamber, the second chamber pre-filled with a flushing fluid;
a nozzle disposed at a distal end of the body and in fluid communication with the vascular access device, the nozzle defining a first conduit in fluid communication with the first chamber, and a second conduit in fluid communication with the second chamber;
a first plunger slidably engaged with the first chamber and configured to be withdrawn proximally to draw a fluid from the first conduit into the first chamber;
a second plunger slidably engaged with the second chamber and configured to urge the flushing fluid through the second conduit; and
a locking mechanism including a pawl engaged with the second plunger in a locked position and configured to inhibit movement thereof, the locking mechanism configured to transition to an unlocked position when the fluid fills at least a portion of the first chamber.

2. The flushing system according to claim 1, wherein the locking mechanism further includes an aperture disposed proximally of the portion of the first chamber and communicating with a well defined by the body, the aperture configured to provide a fluid flow from the first chamber into the well that causes the pawl to disengage from the second plunger.

3. The flushing system according to claim 2, wherein the fluid flow within the well causes the pawl to disengage the second plunger under a gravity flow.

4. The flushing system according to claim 2, wherein the locking mechanism further includes a float actuator configured to transition to an unlocked position when the fluid from the first chamber enters the well.

5. The flushing system according to claim 4, wherein the float actuator includes a one or more of a balloon, air space, or second material that is less dense than the fluid within the first chamber.

6. The flushing system according to claim 4, wherein the float actuator is pivotably coupled to an inner surface of the well and configured to pivot between the locked position and the unlocked position.

7. The flushing system according to claim 4, wherein the locking mechanism further includes a pushrod slidably engaged with the well and transitionable between a locked position and an unlocked position, in the locked position, the pushrod engages the pawl and prevents the pawl from disengaging the second plunger, in the unlocked position the pushrod disengages the pawl to allow the pawl to disengage the second plunger.

8. The flushing system according to claim 7, wherein the float actuator in the locked position engages the pushrod to prevent the pushrod from transitioning to the unlocked position, and the float actuator in the unlocked position allows the pushrod to slide to the unlocked position.

9. A method of flushing an implanted catheter, comprising:
withdrawing a first plunger, slidably engaged with a first chamber of a body, to draw a predetermined volume of fluid through a nozzle and into the first chamber;
transitioning a locking mechanism to an unlocked position, using the fluid within the first chamber, to disengage the locking mechanism from a second plunger; and
sliding the second plunger distally to urge a flushing solution that is prefilled within a second chamber of the body through the nozzle and into the implanted catheter.

10. The method according to claim 9, wherein subsequent to the predetermined volume of fluid entering the first chamber, flowing a portion of the fluid from the first chamber into a well, defined by the body, to actuate the locking mechanism and disengage a pawl from the second plunger.

11. The method according to claim 10, wherein the portion of the fluid from the first chamber actuates the pawl under a gravity flow.

12. The method according to claim 10, wherein the portion of the fluid within the well actuates a float actuator from a locked position to an unlocked position.

13. The method according to claim 12, wherein the float actuator includes a portion that is less dense than the portion of the fluid within the well.

14. The method according to claim 12, further including transitioning the float actuator and a pushrod to an unlocked position that transitions the pawl to the unlocked position.

15. The method according to claim 9, wherein the fluid in the first chamber is blood and the flushing solution in the second chamber includes one of water, saline solution, or Ringers solution.

16. The method according to claim 9, wherein the nozzle defines a first conduit in fluid communication with the first chamber and a second conduit in fluid communication with the second chamber.

17. The method according to claim 9, wherein the nozzle is configured to engage an access needle that is configured to provide fluid communication with the implanted catheter.

18. A catheter and flushing system, comprising:
a catheter extending between a proximal end and a distal tip; and
a flushing system, comprising:
a body defining a first chamber and a second chamber;
a first plunger slidably engaged with the first chamber;
a second plunger slidably engaged with the second chamber, the second chamber prefilled with a flushing solution; and
a locking mechanism configured, in the locked position, to prevent the second plunger from moving, and configured to transition to an unlocked position to allow the second plunger to move when a predetermined volume of fluid is disposed within the first chamber.

19. The catheter and flushing system according to claim 18, wherein the predetermined volume of fluid within the first chamber is greater than a volume disposed within the catheter between the proximal end and the distal tip.
